(19) United States
(12) Patent Application Publication
(10) Pub. No.: US 2025/0302513 A1
(43) Pub. Date: Oct. 2, 2025

(54) Sterile packaging for an implant and procedure for removing an implant from a sterile packaging

(71) Applicant: Karl Leibinger Asset Management GmbH & Co. KG, Mühlheim an der Donau (DE)

(72) Inventors: Jörg Henke, Mühlheim an der Donau (DE); Martin Pabst, Mühlheim an der Donau (DE)

(21) Appl. No.: 19/097,547

(22) Filed: Apr. 1, 2025

(30) Foreign Application Priority Data
Apr. 2, 2024 (DE) 102024109156.8

(51) Int. Cl. A61B 17/86

(52) U.S. Cl. CPC A61B 17/865

(57) ABSTRACT

A sterile packaging for a sterile object includes an outer packaging (1), an outer container (10) arranged in the outer packaging (1) that forms a first sterile barrier and in which a first receiving compartment (13) is formed, and an inner container (20) arranged in the first receiving compartment (13) and that forms a second sterile barrier and in which a second receiving compartment (23) is formed. The outer container (10) includes a first tray (15) closed by a peel-off first cover (14), and the inner container (20) include a second tray (25) closed by a peel-off second cover (24). A second recess of the second tray (25) includes an indentation (26) that rests on a surface of a first recess of the first tray (15) in the first receiving compartment (13) as a pivotal point for a tilting movement of the inner container (20) relative to the outer container (10).

### CROSS-REFERENCE TO RELATED APPLICATION

The present application is related and has right of priority to German Patent Application No. DE102024109156.8 filed on Apr. 2, 2024, which is incorporated by reference in its entirety for all purposes.

### TECHNICAL FIELD

The present invention relates generally to a sterile packaging for an implant and to a procedure for removing an implant from a sterile packaging.

### BACKGROUND

Conventional sterile packaging can be difficult to handle. Thus, there is a risk of contamination of a sterile implant contained therein, which can disrupt procedures during surgery on a patient. To minimize this risk, sterile packaging having a double sterile barrier is known.

For example, EP 3 760 155 B1 discloses a double packaging for a sterile object, comprising a hollow outer container and an inner container arranged therein, in which the object is accommodated. A membrane that deforms under pressure is provided to seal the outer container.

A further double sterile packaging arrangement for an orthopaedic object is described in EP 3 181 060 B1. An outer tray with a first indentation houses an inner tray with a second indentation. The outer tray and the inner tray are each closed by a lid. In the second indentation, a protrusion for attaching a medical object is arranged on an elevation.

When handling the known sterile packaging, there may be a risk of contamination. There is thus a need to provide a sterile packaging with a double sterile barrier that is particularly easy to use, so that the risk of contamination of a sterile object can be minimized.

### BRIEF SUMMARY

According to example aspects of the present invention, a sterile object can be packaged in a sterile packaging having a double sterile barrier, wherein the sterile object can be particularly easily unpacked, so that the risk of contamination of the sterile object can be minimized.

The sterile packaging for a sterile object includes an outer packaging. The outer packaging is the outermost cover of the sterile packaging and does not have to form a sterile barrier.

The outer packaging can be opened by depressing a predetermined breaking line located at an end section of the outer packaging, for example with a user's finger and raising a lid.

An outer container, which forms a first sterile barrier, is arranged in the outer packaging. The outer container can, for example, be easily removed by simply pulling from the outer packaging.

The outer container includes a first tray with a first recess, which is sealed by a peel-off first lid. A first receiving compartment is incorporated in the first recess.

The outer container can be held at a first end section for opening with a first hand, in particular with the thumb and index finger, whereby the first lid is pulled off with the other hand starting from a second end section of the outer container that is opposite to the first end section. When doing so, the first sterile barrier is opened.

The first receiving compartment contains an inner container which forms a second sterile barrier. During and after pulling off the first lid, the inner container can continue to be kept sterile with the first hand on the first end section of the outer container, so that, for example, unintentional falling out of the inner container can be avoided. In this condition, a transfer of the sterile inner container to a second person, who is specifically in a sterile area such as an operating room or similar, can take place.

The inner container includes a second tray with a second recess, which is closed by a peel-off second lid. A second receiving compartment is incorporated in the second recess.

According to example aspects of the invention, the second recess features an indentation that rests on a surface of the first recess in the first receiving compartment as a pivotal point for a tilting movement of the inner container relative to the outer container.

By applying pressure to the inner container while holding the opened outer container at the first end section, the inner container tilts around the pivot relative to the outer container, allowing the inner container to be removed without touching the outer container. For this purpose, the inner container can be grasped at a first end section opposite the pivotal point. The tilting motion thus enables particularly safe and easy removal of the sterile inner container from the outer container. Thus, contamination of the inner container can be avoided.

The sterile object or a carrier for the sterile object can be arranged in the second receiving compartment. Removal of the carrier, respectively the sterile object, from the inner container is performed in a manner similar to the removal of the inner container from the outer container by holding the inner container at the first end section and pulling off the second lid, starting from a second end section of the inner container that is opposite the first end section. Then the sterile object or carrier can be removed easily and safely. The inner container is usually opened in a sterile environment.

According to a preferred example embodiment, the first tray includes a peripheral edge section that surrounds the first recess and to which the removable first lid is attached.

In a similar manner, the second tray can have a peripheral edge section that surrounds the second recess and to which the removable second lid is attached.

The preferred edge section of the first tray and the second tray forms a flat surface framing the respective recess for attaching the respective lid. Furthermore, shaping of the edge section around the recess can increase the rigidity of the first and second trays respectively.

Preferably, the sterile packaging is essentially in the form of a cuboid that extends in a longitudinal direction. The outer container preferably features a suitably fitting shape so that the outer container can be inserted precisely into the outer packaging. With particular preference, additional space can be provided in the outer packaging for a package insert or similar.

The first tray preferably features a first end section for retaining the outer container and a second end section opposite the first end section in longitudinal direction. The end sections can each be formed as part of the preferred edge section. The end sections are designed in a suitable manner for easy grasping of the outer container between the thumb and index finger.

The first recess is preferably formed in longitudinal direction between the first end section and the second end section. Furthermore, a less pronounced section can be formed between the second end section and the first recess to allow the second end section to be kinked. For example, the thickness of the tray material can be reduced in the less pronounced section to allow easy kinking or bending of the second end section. Removal of the inner packaging can be further simplified by bending, respectively kinking, the second end section. Furthermore, kinking can simplify removing of the first lid.

Similarly, the inner packaging can also feature a less pronounced section for kinking a corresponding second end section. On the one hand, this can make it easier to remove the second lid. On the other hand, it can make it easier to remove the sterile object or carrier from the inner packaging.

According to a preferred example embodiment, the outer packaging can be designed as a cardboard box. Preferred in particular, the outer packaging can feature a predetermined breaking line for opening the outer packaging. The predetermined breaking line can, for example, be designed as a perforation that runs around at least part of the circumference of the outer packaging, so that breaking the perforation creates a removable or hinged lid. To make it easier to break the perforation, a cut-out or perforated pressure point can be formed on the surface of the outer packaging in the vicinity of the predetermined breaking line. Furthermore, the outer packaging can preferably feature a transparent window through which the contents of the outer packaging can be seen.

According to a preferred example embodiment, a carrier is arranged in the second receiving compartment, which features a shaped part with a third receiving compartment for the sterile object. For example, the molded part can be designed to exert a clamping effect on the sterile object to keep it in place. This can prevent the sterile object from accidentally falling out of the molded part. In preferred example embodiments, the sterile object may be, in particular, a bone screw or the like.

The carrier preferably features a sloping surface at one end section, whereby an end area of the sloping surface rests as a pivot point for a tilting movement of the carrier relative to the inner container on a surface of the second recess in the second receiving compartment. Thus, the carrier can be tilted from the outer packaging in a similar manner to the inner packaging by applying pressure to the inner packaging, while the carrier is held in place by pressure. This thus ensures that the carrier can be removed from the inner packaging particularly easily and safely.

According to a particularly preferred example embodiment, the outer packaging of the sterile packaging can be in the form of a prism that extends along a longitudinal direction. Such a prism-shaped outer packaging includes two triangular lateral faces that are opposite each other along the longitudinal direction and form a base and a top face of the prism. Furthermore, this outer packaging includes three rectangular lateral faces that form one lateral surface area of the prism. The bottom face and top face may, in particular, be arranged perpendicular to the longitudinal direction.

A multitude of such prism-shaped outer packagings can be stacked in a particularly space-saving manner. Thus, for example, a large number of such sterile packagings can be packed in a carton or similar, or stored in a storage location.

The bottom face and the top face of the prism can preferably have the shape of a right-angled triangle. Thus, two such prism-shaped outer packagings can be stacked on top of each other to form a rectangular base. Consequently, such sterile packagings can be stored in a particularly space-saving manner.

The outer container preferably features a corresponding cross-sectional shape that corresponds to the triangular cross-sectional shape of the prism-shaped outer packaging. In particular, the first recess of the first tray may feature a corresponding substantially triangular cross-section, the apex of which may be rounded. Similarly, the second tray of the inner packaging can feature a cross-section that is designed to save as much space as possible in the first receiving compartment.

According to an alternative example embodiment, the bottom face and the top face of the prismatic form of the outer packaging feature the shape of a rectangular prism. Hereby, two opposite lateral lengths of the bottom face and the top face are of different lengths, so that one of the lateral lengths is at least three times as long as the other of the lateral lengths. Also, with such an example embodiment, the sterile packagings can be stored in a particularly space-saving manner.

### BRIEF DESCRIPTION OF THE DRAWINGS

Example embodiments of the invention are described in detail on the basis of the figures. Illustrated in:

FIG. 1 a perspective view of an outer packaging;

FIG. 2 a perspective view of an outer container;

FIG. 3 a perspective view of an inner container with sterile objects;

FIG. 4 a perspective view of a prism-shaped outer packaging;

FIG. 5 a perspective view of the prism-shaped outer packaging, as well as the corresponding outer container and inner container;

FIG. 6 a lateral view of an outer container with a tilted inner container;

FIG. 7 a perspective view of an inner container with a tilted carrier retaining a sterile bone screw;

FIGS. 8A, 8B, and 8C Steps S1 to S13 of a procedure for removing a bone screw from a sterile packaging;

FIG. 9 a sectional view through the outer container with tilted inner container;

FIG. 10 a sectional view through the inner container with tilted carrier; as well as

FIG. 11 a perspective view of a further example embodiment of the outer packaging.

### DETAILED DESCRIPTION

Reference will now be made to embodiments of the invention, one or more examples of which are shown in the drawings. Each embodiment is provided by way of explanation of the invention, and not as a limitation of the invention. For example, features illustrated or described as part of one embodiment can be combined with another embodiment to yield still another embodiment. It is intended that the present invention include these and other modifications and variations to the embodiments described herein.

The accompanying figures are intended to provide a further understanding of the embodiments of the invention. They illustrate embodiments and, in the context of the description, serve to explain the principles and concepts of the invention. Other embodiments and many of the advantages mentioned arise with regard to the drawings. The elements of the drawings are not necessarily drawn to scale in relation to each other.

In the figures of the drawing, the same, functionally identical and similarly acting elements, features and components are each provided with the same reference signs-unless otherwise stated.

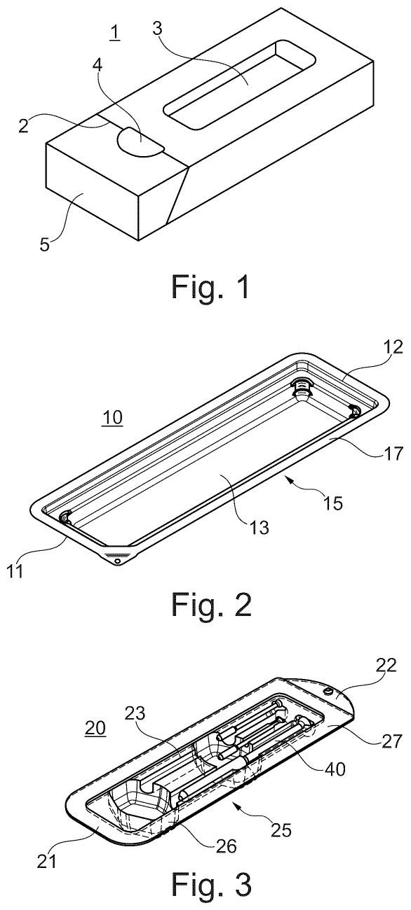

FIG. 1 shows a perspective view of an outer packaging 1 of a sterile packaging according to example aspects of the invention. The outer packaging 1 features a perforated predetermined breaking line 2 that is broken by pressing at the pressure point 4 to open the lid 5. In the example shown, the predetermined breaking line 2 extends along three faces of the outer packaging 1, so that the lid 5 remains hinged to the fourth side. Furthermore, the outer packaging 1 features a window 3 into which a transparent film is adhesive-bonded. This allows the contents of the outer packaging 1 to be viewed. The illustrated outer packaging 1 is cuboid.

FIG. 2 shows a perspective view of an outer container 10 that can be placed in the outer packaging 1. The dimensions of the outer container 10 correspond to the space inside the outer packaging 1 in a suitable manner.

The outer container 10 is depicted without a lid in FIG. 2. The outer container 10 includes a first tray 15 with a first recess, which forms a first receiving compartment 13. The first tray 15 can, for example, be manufactured as a low-cost plastic injection-molded part or by a suitable embossing process. A peripheral edge section 17 is formed around the first recess, which increases the strength of the first tray 15. A first end section 11 and a second end section 12 each are formed at each of the two ends in the longitudinal direction of the tray 15.

FIG. 3 shows a perspective view of an inner container 20 in which three sterile screws 40 are arranged. The inner container 20 can be housed in the outer container 10. The dimensions of the inner container 20 correspond to the space in the first receiving compartment 13 in a suitable manner.

The inner container 20 is depicted without a lid in FIG. 3. The inner container 20 includes a second tray 25 with a second recess, which forms a second receiving compartment 23. Similar to the first tray 15, the second tray 25 can be manufactured as a low-cost plastic injection-molded part or by a suitable embossing process or similar. A peripheral edge section 27 is formed around the second recess, which increases the strength of the second tray 25. A first end section 21 and a second end section 22 each are formed at each of the two ends in the longitudinal direction of the second tray 25.

The sterile screws 40 are clamped in molded parts of the tray 25 so that the sterile screws 40 cannot fall out easily. The screws 40 can be tilted by bending the tray 25, to allow easy removal.

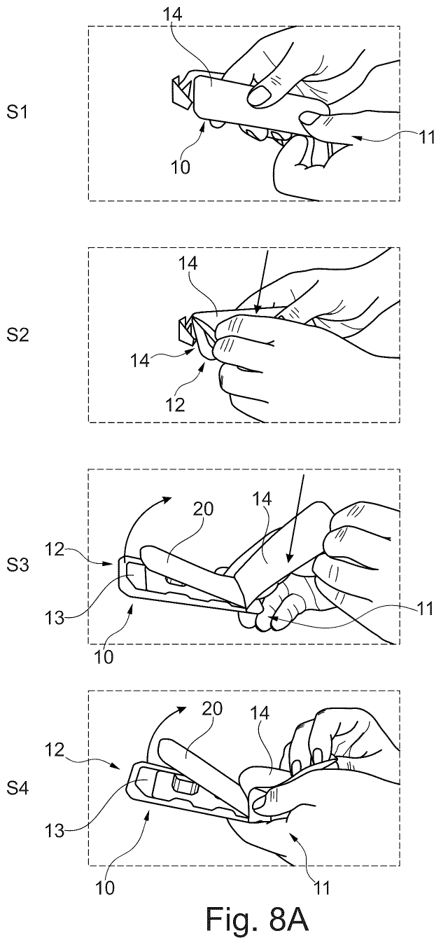

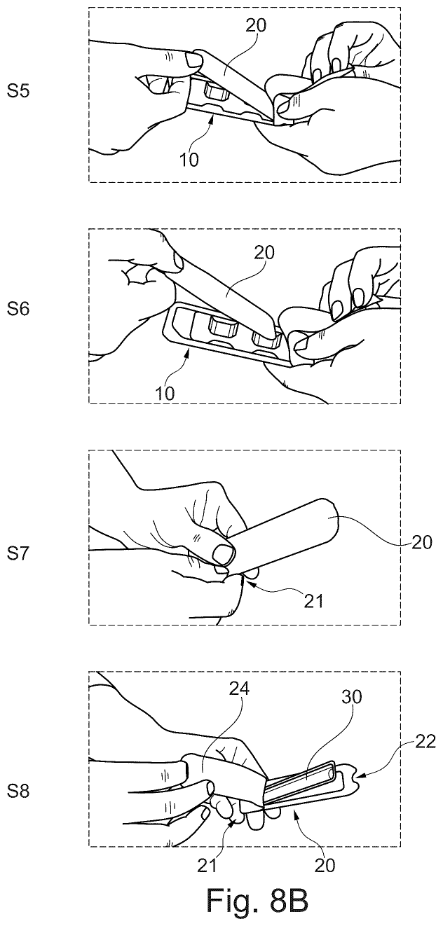

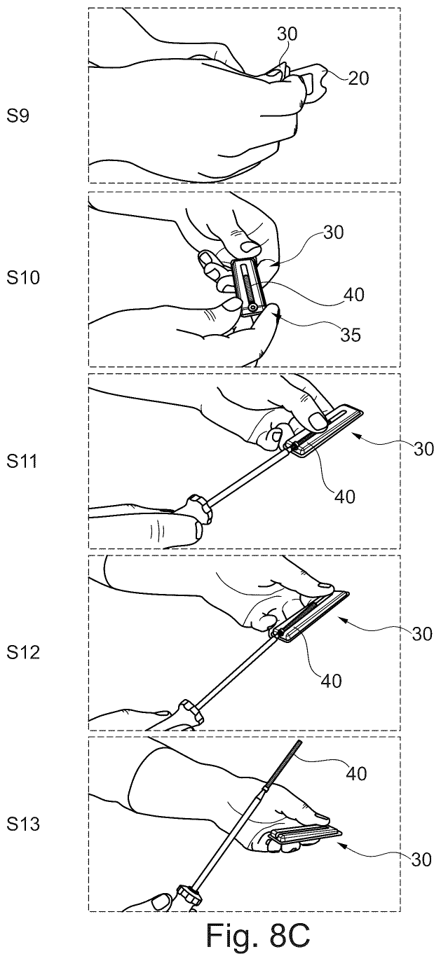

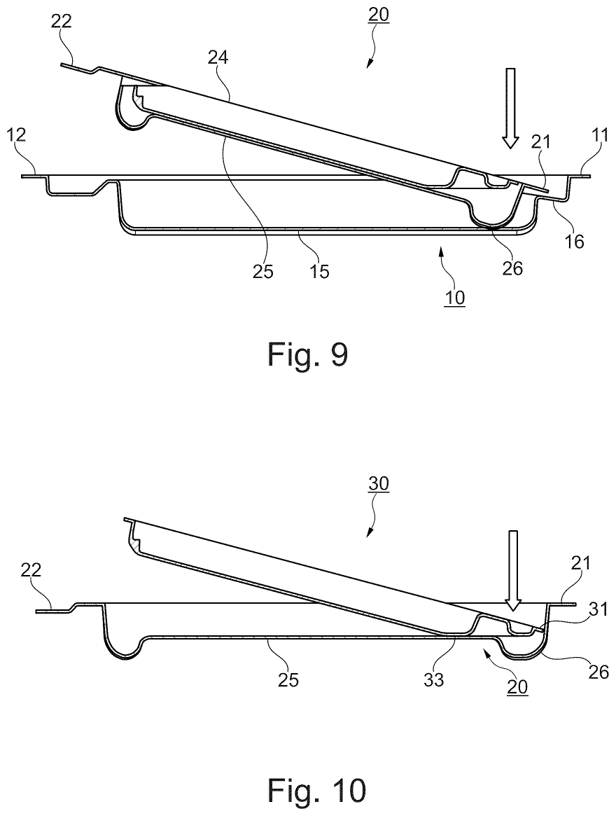

The second recess further features an indentation 26 (see FIG. 9) that rests on a surface of the first recess in the first receiving compartment 13 as a pivotal point for a tilting movement of the inner container 20 relative to the outer container 10 if the inner container 20 is arranged in the first receiving compartment 13. By applying pressure to the first end section 21, the inner container 20 can be tilted against the outer container 10 so that the inner container 20 can be removed particularly easily. This procedure is described in more detail in FIGS. 8A-8C.

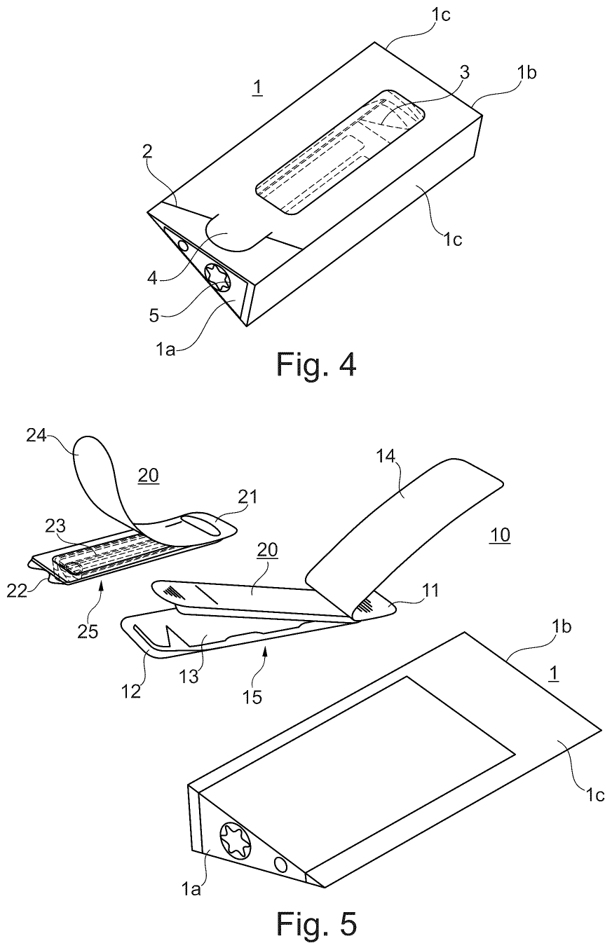

FIG. 4 shows a perspective view of prism-shaped outer packaging 1 according to a preferred example embodiment of the sterile packaging according to the invention. Similar to the cuboid outer packaging 1 shown in FIG. 1, the prism-shaped outer packaging 1 features a predetermined breaking line 2, a window 3, a pressure point 4 and a hinged lid 5. Different from FIG. 1, the prism-shaped outer packaging 1 features a triangular cross-section. In other words, the bottom face 1a and the top face 1b of the prism have the shape of a right-angled triangle. Consequently, a large number of such sterile packagings can be stacked or packaged and stored in a particularly space-saving manner.

FIG. 5 shows a perspective view of the prism-shaped outer packaging 1 as well as a corresponding outer container 10 and inner container 20. The outer container 10 and the inner container 20 are similar to those in FIGS. 2 and 3, whereby those in FIG. 5 feature a cross-sectional shape corresponding to the triangular cross-section of the outer packaging 1.

The center view of FIG. 5 shows the outer container 10 with the lid 14 partially removed from the second end section 12 in the direction of the first end section 11. The inner container 20 is shown in a tilted position in which the sealed sterile inner container 20 can be removed particularly easily and without contamination from the outer container 10. By holding the outer container 10 at the first end section 11 and pressing on the first end section 21 of the inner container 20, the inner container 20 tilts around a pivotal point. An indentation 26, for example, can serve as a pivotal point, resting on a surface in the receiving compartment 13 of the outer container 10.

At the top left, FIG. 5 shows the inner container 20 with the lid 24 partially removed from the second end section 22 in the direction of the first end section 21, so that a second receiving compartment 23 can be seen. In the second receiving compartment 23, a carrier for a sterile object or the sterile object without a carrier can be arranged.

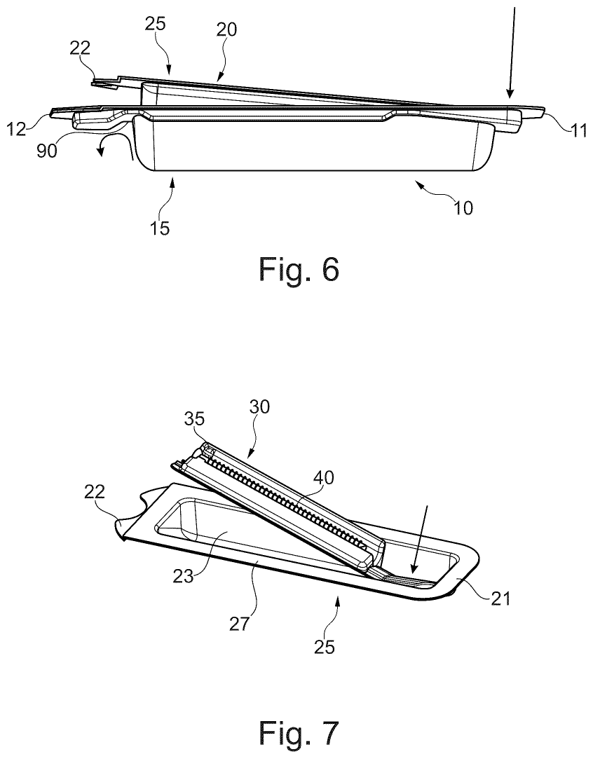

FIG. 6 shows a side view of the outer container 11 from FIG. 5 with a tilted inner container 20. A representation of the lid 14 was omitted in FIG. 6. The arrow indicates the direction of a force for exerting pressure on the first end section 11. This force also acts on the first end section 21 of the inner container 20 and causes the inner container 20 to tilt relative to the outer container 10, so that the inner container 20 partially protrudes out of the outer container 10. Thus, the sterile inner container 20 can be easily removed from the outer container 10 by grasping the second end section 22.

The curved arrow in FIG. 6 illustrates a kinking movement of the second end section 12 at a thinned section of the first tray 15. For example, a user grasps the first end section 11 with the thumb and index finger of the right hand and the second end section 12 with the thumb and index finger of the left hand, tilting the inner container 10 outwards by applying pressure between the thumb and index finger of the right hand and bending the second end section 12 downwards. This way, a second person in a sterile area can easily grasp and remove the sterile inner container 20 with a minimal risk of contamination.

The first tray 15 features a peripheral edge section 17. In addition, a bead for grasping is formed at both end sections 11 and 12, which borders on the edge section. An inner surface at the first end section 11 is inclined at a shallow angle of about ten (10) to thirty (30) degrees, preferably approx. fifteen (15) to twenty-five (25) degrees, to define the tilted position of the inner container 20 relative to the outer container 10.

FIG. 7 shows a perspective view of the interior container 20 with a tilted carrier 30 retaining a sterile bone screw 40. To hold the bone screw 40, the carrier 30 features a molded part 35 in which the bone screw 40 is clamped to prevent the bone screw 40 from falling out.

The carrier 30 can be accommodated in a second receiving compartment 23 of the second tray 25. In FIG. 7, the carrier 30 is tilted relative to the inner container 20 by applying pressure to the first end section 22. For this purpose, the carrier 30 can feature a sloped surface at a corresponding first end section, which rests on an inner surface of the receiving compartment 23 in the tilted state. The sloped surface thereby defines a tilt angle for the carrier 30 relative to the inner container 20.

FIG. 9 shows a sectional view through the outer container 10 with the inner container 20 tilted, similar to FIG. 6. FIG. 10 shows a sectional view through the inner container 20 with the carrier 30 tilted, similar to FIG. 7.

In FIG. 9, the indentation 26 of the second recess can be seen, which rests as a pivotal point for the tilting movement of the inner container 20 relative to the outer container 10 on the surface of the first recess in the first receiving compartment 13. By pressing (see arrow in FIG. 9) on the first end section 21 of the inner container, the opposite second end section 22 tilts out of the outer container 10. The tilting movement takes place around the indentation 26, which rests on the bottom of the recess 15 of the outer container 10.

Furthermore, the outer container 10 features a sloped surface 16 on the first end section 11, which serves as a stop for the first end section 21 of the inner container 20 and defines an end position of the tilting movement, so that the tilted inner container 20 assumes a predetermined and stable tilted position, which facilitates the removal of the inner container 20 and prevents the inner container 20 from dropping down.

Tilting the carrier 30 out of the inner container 20 works in a similar manner. By pressing on the first end section 21 of the inner container 20 and on the first end section 31 of the carrier 30, the carrier performs a tilting movement around one end of a sloped surface 33 at the base of the carrier 30. The end of the sloped surface 33 rests on the base in the recess 25 of the inner container 20 and serves as a pivot for the tilting movement. The sloped surface 33, similar to the sloped surface 16 of the outer container 10, defines an end position of the tilting movement so that the carrier 30 can be held securely and stably in the tilted position.

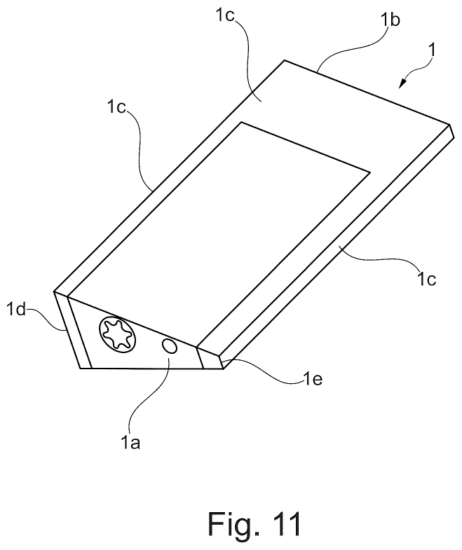

FIG. 11 shows a further example embodiment of the outer packaging 1 of the sterile packaging. In this example embodiment, the outer packaging features the shape of a prism, which features two square side faces 1a, 1b. The lateral face 1a forms the bottom face of the prism. The lateral face 1b forms the top face of the prism. The lateral surface area is formed by four rectangular and right-angled lateral faces 1c. Two opposing face lengths 1e, 1d of the bottom face 1a have different lengths.

FIGS. 8A, 8B and 8C illustrate steps S1 to S14 of a procedure according to example aspects of the invention for removing a bone screw 40 from the sterile packaging according to example aspects of the invention, as shown in FIGS. 1 to 7.

Not shown is a step for opening the outer packaging 1 by pressing in the predetermined breaking line 2 (see FIG. 1 and FIG. 4), which is arranged at one end section of the outer packaging 1, with a user's finger and raising a lid 5 of the outer packaging.

The step of withdrawing the outer container 10, which is located in the outer packaging 1, from the outer packaging 1 is also not shown. Withdrawal is performed, for example, in the longitudinal direction of the sterile packaging.

In step S1, the user has already withdrawn the outer container 10 from the outer packaging 1. The lid 14 is still closed. Thus, the first sterile barrier is intact. The outer container 10 can be handled without risk.

In step S2, the user grasps the first end section 11 of the outer packaging 10 between the thumb and index finger of the right hand and an opposite corner of the lid 14 at the second end section 12 with the left hand.

In step S3, the user continues to hold the outer container 10 at the first end section 11 and pulls the first lid 14 open starting from the second end section 12 of the outer container 10. In doing so, the user exerts pressure on the first end section 11. The pressure acts on the inner container 20, which is located in the first receiving compartment 13, so that the inner container 20 tilts outwards from the outer container 10 via a pivotal point that rests on an inner surface of the receiving compartment 13.

In step S4, the inner container 20 is tilted up as far as possible out of the first receiving compartment 13 of the outer container 10. When doing so, the user pulls the lid 14 backwards in direction of the first end section 11 to avoid contaminating the sterile inner container 20. Here, the second end section 12 can be creased downwards to make it easier to remove the inner container 20.

In step S5, a second user grasps the inner container 20 at a first end section 21 opposite the pivotal point. In step S6, the second user has removed the inner container 20.

In step S7, the second user holds the inner container 20 between the thumb and index finger of the left hand at the first end section 21. In step S8, he removes the second lid 24 with his right hand, starting from a second end section 22 of the inner container 20 that is opposite the first end section 21, thereby opening the second sterile barrier. The pressure applied to the first end section 21 causes the sterile carrier 30, which is located in the second receiving compartment 23, to tilt outwards.

In step S9, the second user can grasp the sterile carrier 30 with the right hand and remove the sterile carrier 30 from the inner container 20.

As shown in S10, the sterile carrier 30 holds a sterile bone screw 40 clamped in a molded part 35. As shown in S11, the screw 40 can be held securely with the carrier 30 while a screwdriver is inserted into the head of the screw 40.

As shown in S12, the screw 40 can be loosened by tilting the screw 40 slightly out of the carrier 30. In step S13, the screw 40 loosened from the carrier 30 is held by the screwdriver.

The procedure illustrated here thus enables a sterile screw to be removed safely and without contamination from a sterile packaging with two sterile barriers. The clamping effect of the molded part can prevent the screw from falling out of the carrier. The respective tilting function enables the sterile contents to be transferred or removed without contamination. Of particular advantage here is that tilting can be generated by pressure when holding, so that particularly simple and safe handling is achieved.

The terms “left hand” and “right hand” are not meant to be restrictive, but are merely intended as examples. It is understood that the user's other hand can also perform the respective task in each case.

In the present description, several features have been labeled with the terms “first” and “second”. These designations only serve to clearly distinguish the individual features. In particular, no spatial or functional arrangement or prioritization should be inferred from this.

If a list of alternatives is followed by “or” in this application, it should be understood that both the listed alternatives by themselves and, if appropriate, a combination of several or all listed alternatives are to be understood.

Modifications and variations can be made to the embodiments illustrated or described herein without departing from the scope and spirit of the invention as set forth in the appended claims. In the claims, reference characters corresponding to elements recited in the detailed description and the drawings may be recited. Such reference characters are enclosed within parentheses and are provided as an aid for reference to example embodiments described in the detailed description and the drawings. Such reference characters are provided for convenience only and have no effect on the scope of the claims. In particular, such reference characters are not intended to limit the claims to the particular example embodiments described in the detailed description and the drawings.

1-11. (canceled)

12. A sterile packaging for a sterile object (40), comprising:
an outer packaging (1);
an outer container (10) arranged in the outer packaging (1) and forming a first sterile barrier, the outer container (10) having a first receiving compartment (13) in the outer container (10); and
an inner container (20) arranged in the first receiving compartment (13) and forming a second sterile barrier, the inner container (20) having a second receiving compartment (23) in the inner container (20),
wherein the outer container (10) comprises a first tray (15) closed by a peel-off first lid (14) and comprises a first recess, the first receiving compartment (13) formed in the first recess of the first tray (15),
wherein the inner container (20) comprises a second tray (25) closed by a peel-off second lid (24) and comprises a second recess, the second receiving compartment (23) formed in the second recess of the second tray (25), and
wherein the second recess comprises a projection (26) that rests on a surface of the first recess in the first receiving compartment (13) and forms a pivotal point for tilting movement of the inner container (20) relative to the outer container (10).

13. The sterile packaging of claim 12, wherein:
the first tray (15) comprises a peripheral edge section (17) that surrounds the first recess and to which the peel-off first cover (14) is attached; and
the second tray (25) comprises a peripheral edge section (27) that surrounds the second recess and to which the peel-off second cover (24) is attached.

14. The sterile packaging of claim 12, wherein:
the sterile packaging extends along a longitudinal direction;
the first tray (15) comprises a first end section (11) for holding the outer container (10) and a second end section (12) opposite the first end section (11) in the longitudinal direction;
the first recess is formed in the longitudinal direction between the first end section (11) and the second end section (12); and
a thinned section (90) is formed between the second end section (12) and the first recess to allow for creasing of the second end section (12).

15. The sterile packaging of claim 12, wherein one or both of:
the outer packaging (1) comprises a predetermined breaking line (2) for opening the outer packaging (1); and
the outer packaging (1) comprises a transparent window (3).

16. The sterile packaging of claim 12, wherein a carrier (30) is arranged in the second receiving compartment (23) and comprises a shaped part (35) with a third receiving compartment for the sterile object (40).

17. The sterile packaging of claim 16, wherein the carrier (30) has an angled surface (33) at one end section (31), and an end region of the angled surface (33) rests on a surface of the second recess in the second receiving compartment (23) to form a pivotal point for a tilting movement of the carrier (30) relative to the inner container (20).

18. The sterile packaging of claim 16, wherein one or both of:
the shaped part (35) exerts a clamping action on the sterile object (40); and
the sterile object (40) is a bone screw.

19. The sterile packaging of claim 12, wherein the outer packaging (1) is formed as a prism with two triangular or rectangular lateral faces (1a, 1b) that are opposite each other along a longitudinal direction and that form a bottom face (1a) and a top face (1b) of the prism and with three or four rectangular lateral faces (1c) forming a lateral surface area of the prism.

20. The sterile packaging of claim 19, wherein the bottom face (1a) and the top face (1b) of the prism are shaped as a triangle and/or the outer packaging (1) is formed from cardboard.

21. The sterile packaging of claim 19, wherein the bottom face (1a) and the top face (1b) are formed as a rectangular prism, and two opposite lateral lengths (1d, 1e) of the bottom face (1a) and the top face (1b) have different lengths such that one of the lateral lengths (1d) is at least three times as long as the other lateral lengths (1e).

22. A method for removing a sterile object (40) from a sterile packaging, comprising:
opening an outer packaging (1) by pressing in a predetermined breaking line (2) that is arranged at one end section of the outer packaging (1) and raising a lid (5) of the outer packaging (1);
withdrawing an outer container (10) from the outer packaging (1), the outer container (10) comprising a first tray (15) closed by a peel-off first lid (14) and having a first recess with a first receiving compartment (13);
holding the outer container (10) at a first end section (11) and pulling off the first lid (14) starting from a second end section (12) of the outer container (10) that is opposite the first end section (11);
applying pressure, while holding the opened outer container (10) at the first end section (11), on an inner container (20) that is arranged in the first receiving compartment (13) and comprises a second tray (25) that is closed by a pull-off second lid (24) and has a second recess with a second receiving compartment (23), the second recess comprising a bulge (26) that rests on a surface of the first recess in the first receiving compartment (13) and forms a pivotal point, the application of pressure tilting the inner container (20) about the pivotal point relative to the outer container (10);
grasping the inner container (20) at a first end section (21) opposite the pivotal point;
holding the inner container (20) at the first end section (21) and pulling off the second lid (24) starting from a second end section (22) of the inner container (20) that is opposite the first end section (21); and
withdrawing the sterile object (40) arranged in the inner container (20), or withdrawing a carrier (30) arranged in the inner container (20) with the sterile object (40).
